(19) United States
(12) Patent Application Publication
(10) Pub. No.: US 2026/0076530 A1
(43) Pub. Date: Mar. 19, 2026

(54) NORMALIZED SPECTRAL ANALYSIS FOR ENDOSCOPE ASSESSMENT

(71) Applicant: STERIS Corporation, Mentor, OH (US)

(72) Inventors: Maciej E. Dybiec, Cooper City, FL (US); Ana K. Ochoa, Pembroke Pines, FL (US); Leighton A. Schonlau, Cooper City, FL (US)

(21) Appl. No.: 19/323,449

(22) Filed: Sep. 9, 2025

Related U.S. Application Data
(60) Provisional application No. 63/696,545, filed on Sep. 19, 2024.

(51) Int. Cl. A61B 1/00; A61B 1/04

(52) U.S. Cl. CPC A61B 1/00057; A61B 1/043; A61B 1/046; A61B 1/00078

(57) ABSTRACT

Examples relate to methods and systems for evaluating near-infrared (NIR) performance of endoscopes. A method includes illuminating an endoscope's entrance pupil with a first radiant flux diffused through a first integrating sphere, the first radiant flux having a first spectrum, measuring a second radiant flux collected by a second integrating sphere at the exit pupil having a second spectrum, and determining transmission by comparing the spectra. The method includes determining a normalization factor for the second spectrum by finding the maximum value of a ratio of output scaled spectrum and input normalized spectrum. The method includes determining transmission through the endoscope as the ratio of the output normalized spectrum sum in an NIR region to the input normalized spectrum sum in the same NIR region.

### CROSS-REFERENCE TO RELATED APPLICATION

This patent application claims the benefit of U.S. Provisional Patent Application No. 63/696,545, filed Sep. 19, 2024, which is incorporated by reference herein in its entirety.

### BACKGROUND

Endoscopy is a medical imaging technique that allows for minimally invasive visual examination of internal organs and body cavities. Advancements in optical technologies have expanded the capabilities of endoscopes, particularly in the near-infrared (NIR) portion of the electromagnetic spectrum. NIR imaging through endoscopes has become increasingly important in various medical applications, including fluorescence endoscopy for cancer detection and other diagnostic procedures.

The imaging performance of endoscopes, especially in the NIR range, is crucial for accurate diagnosis and effective medical interventions. However, evaluating the NIR performance of endoscopes presents several challenges. These challenges stem from the complex optical systems within endoscopes, which can vary significantly in design, including differences in lens diameter, field of view, and other optical characteristics.

Traditional methods of assessing transmission through an endoscope rely on absolute measurements to compare input radiant flux to output radiant flux. However, these can be problematic due to variations in endoscope design and manufacturing tolerances that can create variations in alignment to measurement hardware. A particularly challenging part of the measurement is precisely measuring the input radiant flux. These factors can lead to inconsistencies in performance evaluations across different endoscope models and testing environments.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 and FIG. 2 illustrate a metrology setup for measuring NIR transmission of an endoscope, according to an example.

FIG. 3 illustrates a flowchart of a method for a technique to standardize measurements of NIR transmission spectra, according to an example.

FIG. 4 illustrates a flowchart of a method for a technique to determine a normalization factor for a NIR transmission spectra, according to an example.

FIG. 5 through FIG. 8 illustrate graphs of spectra at different stages of the technique to determine a normalization factor based on the techniques of FIG. 4, according to an example.

FIG. 9 illustrates the results of a technique to determine a NIR transmission measurement, according to an example.

FIG. 10 and FIG. 11 illustrate normalized spectra and NIR performance calculations across different endoscopes, according to an example.

FIG. 12 illustrates a block diagram of a computing machine, according to an example.

### DETAILED DESCRIPTION

As medical imaging technology continues to advance, there is an ongoing need for more accurate and standardized methods of evaluating endoscope performance, particularly in the NIR spectrum. Accordingly, improvements to assessing NIR performance of endoscopes, particularly rigid endoscopes, are presented herein.

Traditional methods of assessing endoscope performance, such as transmission of near-infrared (NIR) light, often rely on absolute measurements of a total radiative flux for quantifying both the input light and the output light. Using absolute measurements as the basis of a transmission measurement can lead to inconsistencies when comparing different endoscope models with varying physical characteristics such as lens diameter, field of view, and other optical properties.

Additionally, as shown in FIG. 2, obtaining the exact value of the radiant flux entering the endoscope (NIR in) is challenging due to variations in endoscope design (e.g., alignment of optics along a mechanical axis) and the inability to fully isolate the input radiant flux that enters the endoscope's field of view. Variations in hardware inconsistencies, instrument response functions, collection fiber transmission fluctuations, etc., all contribute to inconsistencies in comparison of NIR performance for endoscopes.

The described technology addresses these problems by measuring spectra of the incoming and outgoing light, and then using a relative measurement to assess the NIR portion of the spectra. The described technology implements a spectrum normalization technique that normalizes both the incoming and outgoing light spectra to a point of maximum transparency for each endoscope. The technique involves the following: a) normalizing both incoming and outgoing spectra to unity at their respective maximum values, b) calculating the ratio of the normalized outgoing spectrum to the normalized incoming spectrum, c) identifying the maximum value point in this ratio, which corresponds to the wavelength at which the endoscope is most transparent, and d) using this maximum value as the normalization factor for the outgoing spectrum.

By applying this normalization technique, the described technology eliminates the impact of variations in physical characteristics between different endoscope designs. The normalized outgoing spectra allow for comparison of NIR performance across various endoscope models, regardless of their specific optical properties (e.g., lens diameters).

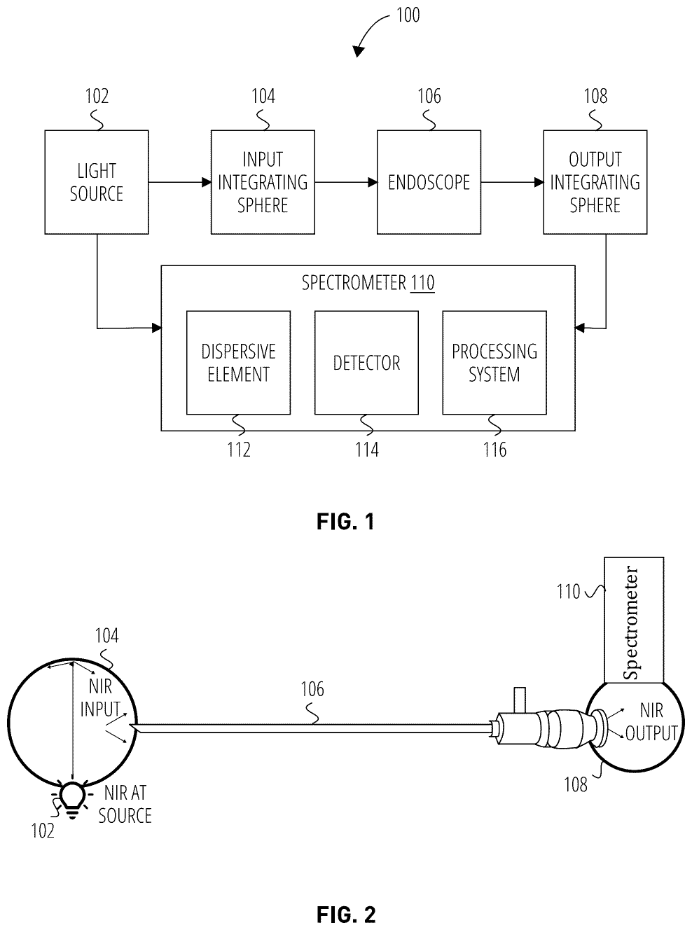

FIG. 1 illustrates a metrology system 100 for measuring NIR transmission of an endoscope. The metrology system 100 includes the basic components and their arrangement for capturing incoming and outgoing light spectra from the endoscope. The metrology system 100 shown in FIG. 1 comprises a light source 102, an input integrating sphere 104, an endoscope 106, an output integrating sphere 108, a spectrometer 110, a dispersive element 112, a detector 114, and a processing system 116.

The light source 102 can be any suitable broadband light source capable of producing light in the visible and near-infrared spectrum. The light from the light source 102 is directed into the input integrating sphere 104, which diffuses the light, ensuring uniform illumination of the endoscope's entrance pupil.

In some examples, the light source can be a fluorescent bulb (with any suitable fluorescence source such as mercury gas, argon gas, xenon gas, and/or a mixture of noble gases, etc.), an incandescent lamp, a tunable laser, and/or any other suitable light source. In some examples, the light source 102 can generate any suitable amount of radiant flux. That is, in some examples, the light source 102 can output any suitable amount of power, in any suitable direction or field of view, as light in the electromagnetic spectrum. In some examples, the light source 102 can have any suitable spectrum, that is, the light source 102 can emit wavelengths across any suitable range of the electromagnetic spectrum. In a particular example, the light source 102 can contain light in the near-infrared (NIR) region of the electromagnetic spectrum, approximately 700 nm to 1000 nm.

The input integrating sphere 104 can be any suitable integrating sphere. In some examples, the light source 102 can be positioned so that radiant flux from the light source 102 enters the input integrating sphere 104 or can be positioned inside of it. The input integrating sphere 104 can have any suitable reflectivity and can diffuse the input light across any suitable range of angles. In some examples, the output side of input integrating sphere 104 can be aligned to the endoscope 106. That is, an entrance pupil of the endoscope 106 can be illuminated with a first radiant flux having a first spectrum using the output side of input integrating sphere 104. In some examples, the input integrating sphere 104 can over-fill the entrance pupil of the endoscope 106.

The endoscope 106 under evaluation is positioned with its entrance pupil facing the output of input integrating sphere 104. The endoscope 106 can be any suitable medical device used to examine the interior of a body cavity or organ. In some examples, the endoscope 106 can be any suitable optical instrument that allows visualization of internal body structures, particularly for medical diagnostic and surgical procedures. In some examples, the endoscope 106 can be used for specialized imaging applications such as fluorescence endoscopy, particularly fluorescence imaging in the NIR region of the electromagnetic spectrum.

In some examples, the endoscope 106 can be a rigid endoscope of various designs, including different diameters, fields of view, or optical magnifications. In some examples, the endoscope 106 can have any suitable additional features that can contribute to imaging applications. For example, the endoscope 106 interior can include additional optical systems, anti-reflection coatings, and/or any other features that specify an endoscope for use in a particular imaging application.

In some examples, the endoscope 106 can have an exit pupil where light exits the endoscope after transmission through the endoscope's optical system. In some examples, the exit pupil comprises any suitable optical system and can have any suitable diameter and/or field-of-view. For example, the exit pupil can include at least one lens that images light and a focal area of interest from an interior of a body cavity or organ.

At the exit pupil of the endoscope 106, an output integrating sphere 108 is positioned to collect the light transmitted through the endoscope. The output integrating sphere 108 ensures that the radiant flux exiting the endoscope, regardless of its angular distribution, is captured for measurement, i.e., measuring the spectrum of the second radiant flux.

The output integrating sphere 108 can be any suitable integrating sphere. The output integrating sphere 108 can have any suitable reflectivity and can collect diffuse light across any suitable range of angles. In some examples, the output side of output integrating sphere 108 can be aligned to the spectrometer 110. That is, in some examples, a radiant flux transmitted through the endoscope 106 can be collected by the output integrating sphere 108 and measured at the spectrometer 110.

Connected to the output integrating sphere 108 is the spectrometer 110. The spectrometer 110 measures the spectral distribution of the light collected by the output integrating sphere 108. In some examples, the spectrometer 110 can be a high-resolution device capable of measuring light intensity across a wide range of wavelengths, including the near-infrared region.

In some examples, the spectrometer 110 can include a dispersive element 112 (e.g., a prism, a diffraction grating, a holographic diffraction grating, etc.) and a detector 114 (or any other suitable opto-electronic detector) to record the dispersed light. In some examples, the spectrometer 110 can include any suitable additional elements (e.g., mirrors, lenses, etc.) to image light input to the spectrometer onto the dispersive element 112 and/or detector 114. In some examples, the dispersive element 112 can be a single element, such as a wide-band grating. In some examples, the dispersive element 112 can be a group of elements, such as multiple gratings (e.g., with increasing precision) mounted on a turret or other adjustable hardware. In some examples, the dispersive element 112 can be responsive to any suitable wavelength band.

In some examples, the detector 114 can be any suitable opto-electronic recording device that is positioned after the dispersive element 112. In some examples, the detector 114 can be a 2D sensor. In some examples, the detector 114 can be a 1D sensor. In some examples, the detector 114 can include any suitable sensor, such as a CCD sensor, a CMOS sensor, etc., of any suitable size.

In some examples, the spectrometer 110 can be controlled by processing system 116. In some examples, the processing system 116 can be any processing system such as machine 1200 (e.g., computer system, computing device, machine, controller, etc.). In some examples, the processing system 116 can include software instructions 1224 that can operate the spectrometer, such as acquiring a spectral image from the detector 114, setting an integration time for the detector 114, binning multiple pixels (in either 1D or 2D) of the detector 114, storing a calibration of the dispersive element 112 to the detector 114, etc. In some examples, images and/or data acquired from the detector 114 can have a calibration (e.g., equation, corrections, etc.) applied based on the dispersive element 112. In some examples, a ‘spectrum’ refers to an image and/or a dataset from the spectrometer 110 that has a calibration applied to it. In some examples, the processing system 116 can perform any other suitable pre-processing steps to an image and/or dataset, such as background subtraction, aberration correction (i.e., to correct artifacts from any optics interior to the spectrometer 110), binning, etc.

In some examples, the input spectrum can include any other suitable corrections, subtractions, etc. For example, the input spectrum can include a calibration factor that accurately maps a position in the dispersive element.

The components of metrology system 100 can be interconnected to allow for the measurement of both the input and output light spectra. The light source 102 can be optically coupled to the input integrating sphere 104, which in turn can illuminate the entrance pupil of the endoscope 106. The exit pupil of the endoscope 106 can be optically coupled to the output integrating sphere 108, which can feed the transmitted light into the spectrometer 110.

In some examples, the setup can include additional components such as optical filters, collimators, or beam splitters to further control or analyze the light. The entire setup can be mounted on an optical bench or similar stable platform to ensure precise alignment and reproducible measurements.

This metrology setup can enable the capture of both the incoming light spectrum (measured at the input integrating sphere 104) and the outgoing light spectrum (measured at the output integrating sphere 108 via the spectrometer 110). These spectra form the basis for the subsequent normalization and analysis processes used to evaluate the NIR performance of the endoscope. In particular, metrology system 100 can be used to determine a transmission measurement through the endoscope 106 by comparing a spectrum of the light source 102 and a spectrum of light transmitted through the endoscope 106.

FIG. 2 illustrates a diagram of the metrology system described in FIG. 1. A light source 102 is diffused through an input integrating sphere 104. While the radiant flux of the light source 102 can be measured at or near the input of the input integrating sphere 104, due to the diffusion of the light source 102 across all angles, the exact value of NIR_IN is difficult to obtain. Thus, an output integrating sphere 108 collects the radiant flux that leaves the endoscope 106 (NIR_OUT). A spectrometer 110 can be used to compare the NIR portions of the input light spectrum and the output light spectrum after a technique to standardize the output spectrum is applied.

Although not shown in FIG. 2, the light source 102 can be optically connected to the spectrometer 110 as described above in FIG. 1.

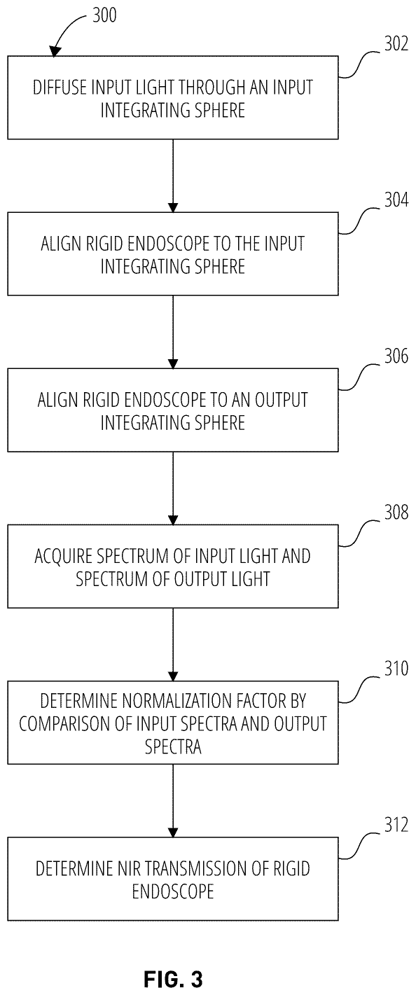

FIG. 3 illustrates a flowchart of a method 300 for a technique to standardize measurements of NIR transmission spectra. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

According to some examples, the method includes diffusing input light through an input integrating sphere at block 302. For example, as shown in FIG. 1, light source 102 can be diffused through input integrating sphere 104.

According to some examples, the method includes aligning a rigid endoscope to the input integrating sphere at block 304. For example, as shown in FIG. 1, the endoscope 106 can be aligned to input integrating sphere 104 so that the entrance pupil of the endoscope 106 is over-filled.

According to some examples, the method includes aligning the rigid endoscope to an output integrating sphere at block 306. For example, as shown in FIG. 1, the endoscope 106 can be aligned to output integrating sphere 108 to collect light at the exit pupil of the endoscope 106 that is transmitted through the endoscope 106.

According to some examples, the method includes acquiring a first spectrum of the input light and a second spectrum of the output light at block 308. For example, as shown in FIG. 1, the spectrometer 110 can be used to acquire a first spectrum of input light (e.g., light source 102) and a second spectrum of light that is transmitted through the endoscope and collected from output integrating sphere 108.

According to some examples, the method includes determining a normalization factor by comparison of input spectra and output spectra at block 310. As a particular example, method 400 described in FIG. 3 can be used to determine the normalization factor.

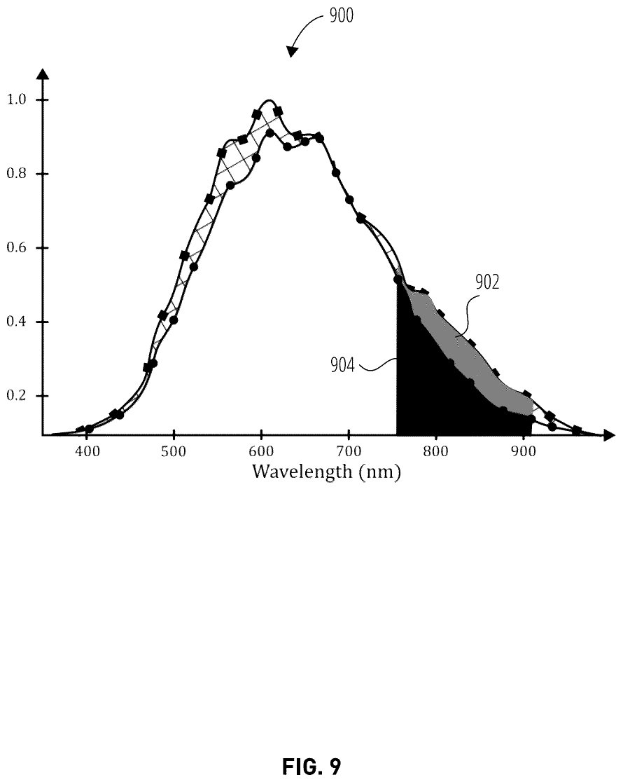

According to some examples, the method includes determining the NIR transmission of rigid endoscope at block 312. In some examples, the NIR transmission can be determined from a comparison or other evaluation of the input spectra and the output spectra. In some examples, spectral intensity values can be integrated across an NIR wavelength range (e.g., 750 nm-1000 nm) for the normalized input spectrum, as is shown below in FIG. 9 for input spectral sum 902. In some examples, normalized spectral intensity values can be integrated across the same NIR wavelength range for the output spectrum with the normalization factor applied, as seen in output spectral sum 904 of FIG. 9. In some examples, the NIR transmission can be the ratio of output spectral sum (e.g., 904) to input spectral sum (e.g., 902), expressed as a percentage value shown in Eq. 1:

$$N I R performance [ \% ] = \frac{N I {R}_{out}}{N I {R}_{in}} \times 100 \qquad Eq . 1$$

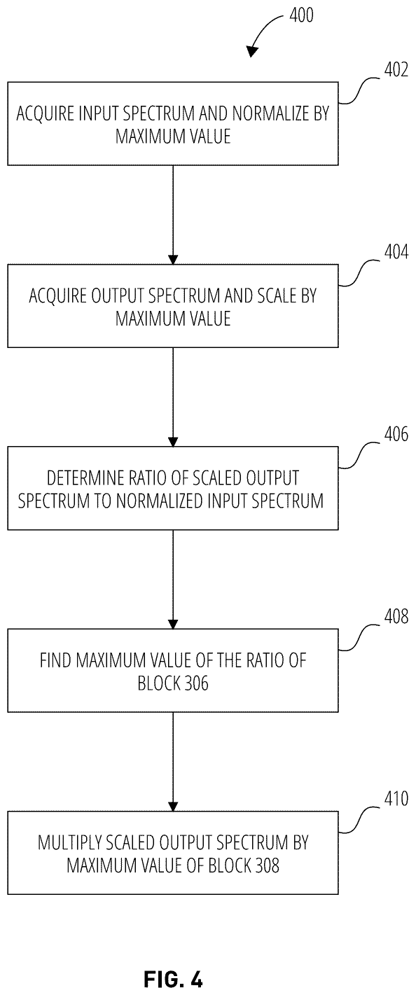

FIG. 4 illustrates a flowchart of a method 400 for a technique to determine a normalization factor for a NIR transmission spectra, according to an example. In some examples, method 400 can receive data, such as spectra, from any other suitable method, such as method 300. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

According to some examples, the method 400 includes acquiring a spectrum of input light (“input spectrum”) and normalizing the input spectrum by its maximum intensity value at block 402. In some examples, the method 400 can acquire the input spectrum from a spectrometer, such as spectrometer 110. In some examples, the method 400 can receive the input spectrum from an operation such as block 308 of method 300.

In some examples, at block 402, a normalized input spectrum can have a maximum value of 1 and a minimum value of 0. In some examples, any other suitable value can be used to normalize the input spectra at block 402. In some examples, for example based on pre-processing (e.g., background subtraction) of the image data from spectrometer 110 at the processing system 116, a normalized input spectrum can have values less than 0, which can be discarded from analysis, rounded to 0, and/or otherwise further processed.

According to some examples, the method 400 includes acquiring an output spectrum and scaling the output spectrum by a maximum intensity value at block 404. In some examples, the method 400 can acquire the output spectrum from a spectrometer, such as spectrometer 110. In some examples, the method 400 can receive the output spectrum from an operation such as block 308 of method 300.

In some examples, scaling the output spectrum can include dividing intensity values of the output spectrum by a maximum intensity value found in the output spectrum. Although such scaling is generally considered to be “normalizing”, as used above for the input spectra, the operations at block 404 use the term “scaled output spectrum” so as to distinguish from the normalization factor determined at block 406 below and further, from the normalized output spectrum generated from applying the normalization factor to the output spectrum as discussed below at block 408. That is, in some examples, the scaled output spectrum determined at block 404 is an intermediate output spectrum.

According to some examples, the method 400 includes determining the ratio of the scaled output spectra to normalized input spectra at block 406. In some examples, this creates a new dataset, the OUT/IN ratio spectrum.

According to some examples, the method 400 includes finding maximum value of the OUT/IN ratio spectrum at block 408. In some examples, the maximum value of the OUT/IN ratio spectrum can be used as a normalization factor for the output spectrum.

According to some examples, the method 400 includes multiplying the scaled output spectra by the normalization factor at block 410. In some examples, the normalization factor is the value found at block 408, that is, the maximum value of the OUT/IN ratio spectrum. In some examples, a normalized output spectrum is the result of applying the normalization factor to the scaled output spectrum. In some examples, an intensity value of the normalized output spectrum is approximately identical to an intensity value of the normalized input spectrum at a particular wavelength. In some examples, the particular wavelength where the normalized output spectrum is approximately identical to the normalized input spectrum is the wavelength value at which the maximum value of the OUT/IN ratio spectrum occurs.

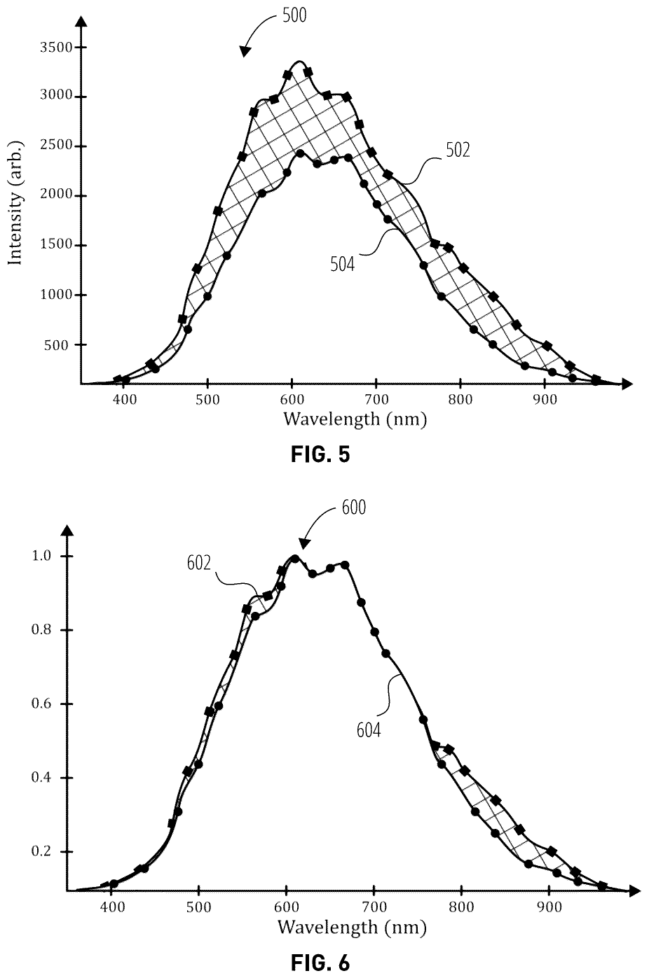

Example spectra showing different stages of spectra during operations of the method 400 can be found in FIG. 5 through FIG. 8.

FIG. 5 through FIG. 8 illustrate graphs of spectra at different blocks of the method 400 to determine a normalization factor according to the techniques of FIG. 4, according to an example.

In each of the FIG. 5 through FIG. 8 (as well as the graphical diagrams in FIG. 9 through FIG. 11), the x-axis of the graphs represent the wavelength of light, typically measured in nanometers (nm). The wavelength range may extend from the visible spectrum (approximately 380-700 nm) into the near-infrared region (typically 700-1400 nm). The y-axis in each graph represents the intensity of light, often measured in arbitrary units (a.u.) or relative intensity. Different graphs can show different intensity units, due to the various calculations performed during the method 400 of FIG. 4.

FIG. 5 is a graphical diagram including spectra 500, specifically, input spectrum 502 and output spectrum 504. The spectra 500 illustrate the spectral distribution of light before and after passing through an endoscope.

Input spectrum 502 represents the spectral distribution of light entering an endoscope's entrance pupil. This spectrum is typically measured at the output of the input integrating sphere 104 in metrology system 100, as described with reference to FIG. 1.

Output spectrum 504 represents the spectral distribution of light exiting the endoscopes' exit pupil, as measured by spectrometer 110 connected to output integrating sphere 108.

The input spectrum 502 generally shows higher intensity values across all wavelengths compared to output spectrum 504. This difference illustrates the light attenuation that occurs as the radiant flux passes through the endoscope's optical system. The attenuation can be due to various factors such as absorption, reflection, and scattering by components of the endoscope's optical system. As seen in FIG. 5, the attenuation can be wavelength dependent and can create specific wavelength regions of output spectrum 504 that are relatively lower than other wavelengths which may show little attenuation. For example, near 500 nm, the output spectrum 504 has less attenuation than around 650 nm. Such attenuation can make it difficult to compare different endoscopes, leading to inconsistencies. A normalization factor as described in method 300 and method 400 can be applied to the input spectrum 502 to compare it to the output spectrum 504, leading to a uniform NIR transmission measurement that can be compared across endoscope designs.

FIG. 6 is a graphical diagram including spectra 600, specifically, normalized input spectrum 602 and a scaled output spectrum 604. In some examples, normalized input spectrum 602 can be determined from input spectrum 502 at block 402 of method 400. In some examples, scaled output spectrum 604 can be determined from output spectrum 504 at block 404 of method 400. As noted above in FIG. 4, the term ‘scaled’ is used to describe the output spectrum divided by its maximum value. A ‘normalized output spectrum’ can be determined from applying a normalization factor to the scaled output spectrum.

As seen in FIG. 6, comparison of the input and output spectra can be challenging when each are normalized by their respective maximum intensity values. For example, while the normalized input spectrum 602 has a maximum value around 600 nm, and the scaled output spectrum 604 also has a maximum value around 600 nm, the output spectrum 504 has less attenuation around 650 nm. This leads to the scaled output spectrum 604 having a greater amount of light in the region around 650 nm than the normalized input spectrum 602, which is not representative of the light propagation in metrology system 100. In some examples, scaled output spectrum 604 is further processed with a normalization factor as described in method 400.

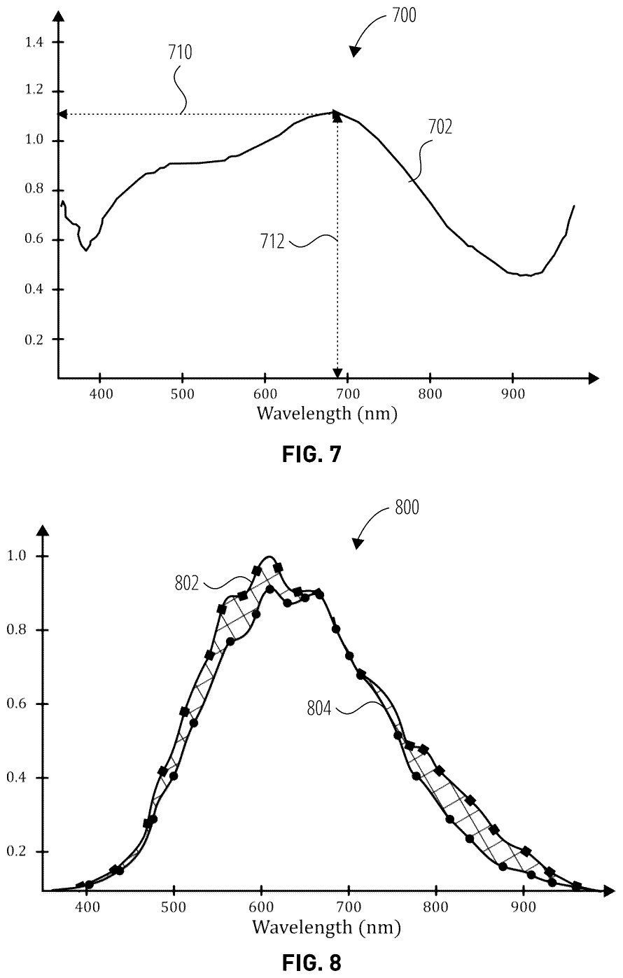

FIG. 7 is a graphical diagram including spectra 700. The spectra 700 comprises a ratio 702, a max value 710, and a wavelength 712. In some examples, ratio 702 is a dataset from computing the ratio of the scaled output spectrum 604 to the normalized input spectrum 602 as described at block 406 of method 400. In some examples, the max value 710 can be the normalization factor applied to scaled output spectrum 604, as seen in FIG. 8.

FIG. 8 is a graphical diagram including spectra 800. The spectra 800 comprises a normalized input spectrum 802, as seen in normalized input spectrum 602, and a normalized output spectrum 804. In some examples, normalized output spectrum 804 can be the scaled output spectrum 604 with the normalization factor applied.

FIG. 9 is a graphical diagram of spectra 900. The spectra 900 comprises an input spectral sum 902 and an output spectral sum 904. In some examples, input spectral sum 902 and output spectral sum 904 can be used to compute the NIR performance of an endoscope. In some examples, the spectral sum can comprise a sum of intensity values of a given spectra, such as normalized input spectrum 802 and/or normalized output spectrum 804. In some examples, input spectral sum 902 and output spectral sum 904 can have any starting wavelength and any ending wavelength.

In some examples, the NIR performance of an endoscope can be calculated as the ratio of the output spectral sum 904 to the input spectral sum 902. In some examples, this ratio can be expressed as a percentage value.

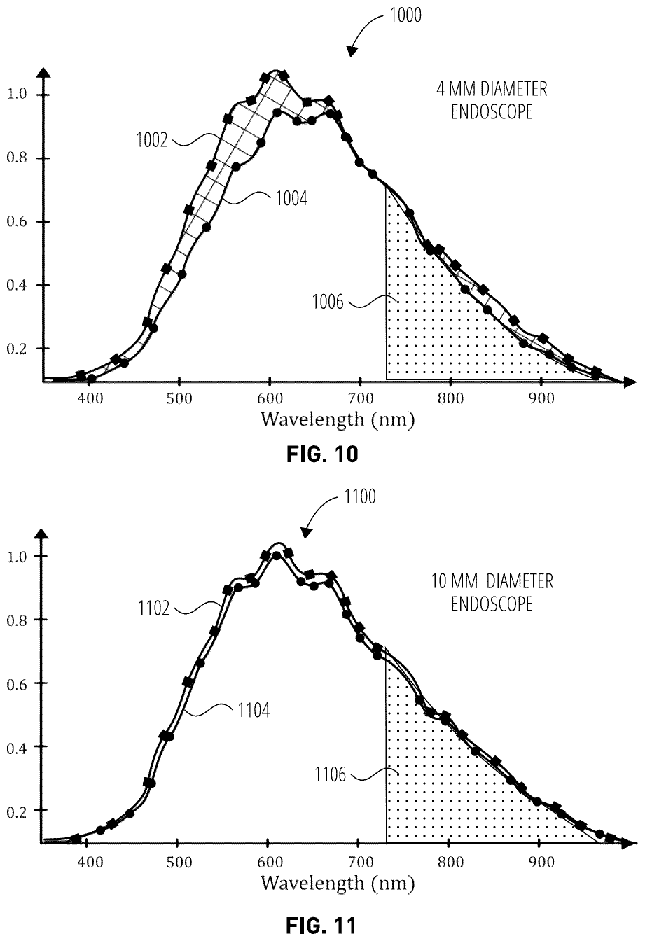

FIG. 10 is a graphical diagram of spectra 1000. The spectra 1000 comprises an input spectrum 1002, a 4 mm output spectrum 1004, and an output spectral sum 1006. In some examples, the 4 mm output spectrum 1004 can be spectrum of the input spectrum that is output from an endoscope having a 4 mm lens at the output side (“the 4 mm diameter endoscope”), such as the model Stryker 502-444-030. In some examples, the 4 mm output spectrum 1004 can have a normalization factor already determined and applied, that is, can be a spectrum determined at block 408 of method 400. In some examples, the ratio of the output spectral sum to the input spectral sum, in the wavelength region shown in output spectral sum 1006, can be 0.875, or 87.5%. In some examples, the NIR transmission of the 4 mm endoscope can be 87.5%.

FIG. 11 is a graphical diagram of spectra 1100. The spectra 1100 comprises an input spectrum 1102, a 10 mm output spectrum 1104, and an output spectral sum 1106. In some examples, the 10 mm output spectrum 1104 can be spectrum of the input spectrum that is output from an endoscope having a 10 mm lens at the output side (“the 10 mm diameter endoscope”), such as the model Stryker 602-103-030. In some examples, the 10 mm output spectrum 1104 can have the normalization factor already determined and applied, that is, can be a spectrum determined at block 408 of method 400. In some examples, the ratio of the output spectral sum to the input spectral sum, in the wavelength region shown in output spectral sum 1106, can be 0.980, or 98.0%. In some examples, the NIR transmission of the 10 mm endoscope can be 98.0%.

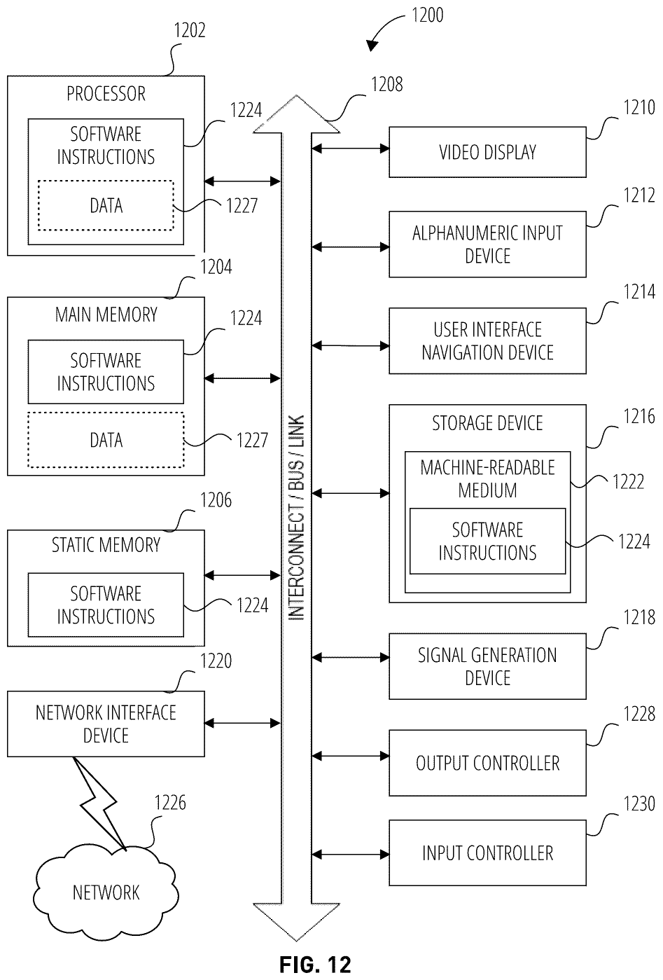

FIG. 12 illustrates a block diagram of an example machine 1200 (e.g., computer system, computing device, machine, controller, etc.) that may be programmed into a special purpose machine suitable for implementing one or more embodiments for data processing, data communication, user interface, or like aspects disclosed herein. For instance, the processing system 116 described above may be embodied by the machine 1200, such as in the form of a computer or specialized electronic device that includes sufficient processing power, memory resources, and communications throughput capability to perform specific compute operations consistent with the examples herein.

The machine 1200 may include a hardware processor 1202 (e.g., a central processing unit (CPU), a graphics processing unit (GPU), a hardware processor core, or any combination thereof), a main memory 1204 and a static memory 1206, some or all of which may communicate with each other via an interconnect, link or bus 1208. The machine 1200 may further include a display unit 1210, an alphanumeric input device 1212 and a user interface (UI) navigation device 1214. In an example, the display unit 1210, alphanumeric input device 1212 and navigation device 1214 may be a touch screen display. The machine 1200 may additionally include a storage device 1216 (e.g., drive unit), a signal generation device 1218 (e.g., an audio or radio signal generation device), and a network interface device 1220 (e.g. for connectivity with a network). The machine 1200 may include an output controller 1228, such as a serial (e.g., universal serial bus (USB), parallel, or other wired or wireless (e.g., infrared (IR), near field communication (NFC), etc.) connection to communicate or control one or more peripheral devices, and an input controller 1230 to connect to more sensors.

The storage device 1216 may include a machine-readable medium 1222 that is non-transitory on which is stored one or more sets of data structures or software instructions 1224 (e.g., software) embodying or utilized by any one or more of the techniques or functions described herein. The instructions 1224 may also reside, completely or at least partially, within the main memory 1204, within static memory 1206, or within the hardware processor 1202 during execution thereof by the machine 1200. In an example, one or any combination of the hardware processor 1202, the main memory 1204, the static memory 1206, or the storage device 1216 may constitute machine readable media.

The term “machine-readable medium” may include any medium that is capable of storing, encoding, or carrying instructions for execution by the machine 1200 and that cause the machine 1200 to perform any one or more of the techniques of the present disclosure, or that is capable of storing, encoding or carrying data structures 1227 used by or associated with such instructions. Non-limiting machine-readable medium examples may include solid-state memories, and optical and magnetic media. Specific examples of machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1224 may further be transmitted or received over a communications network 1226 using a transmission medium via the network interface device 1220 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®), IEEE 802.15.4 family of standards, peer-to-peer (P2P) networks, among others. In an example, the network interface device 1220 may include one or more physical jacks or one or more antennas to connect to the communications network 1226. In an example, the network interface device 1220 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1200, and includes digital or analog communications signals or other intangible medium to facilitate communication of such software.

The devices described herein may be configured to include computer-readable non-transitory media storing computer readable instructions and one or more processors coupled to the memory, and when executing the computer readable instructions configure the machine 1200 to perform steps and operations described above for electronic systems or devices (e.g., to display a user interface and receive user interface commands, perform sensing operations from electromechanical and environmental sensors, extract and identify data values, etc.). The computer-readable non-transitory media includes all types of computer readable media, including magnetic storage media, optical storage media, flash media and solid-state storage media. It should be further understood that software including one or more computer-executable instructions that facilitate processing and operations as described above with reference to any one or all of steps of the disclosure may be installed in and sold with networked devices (e.g., servers or cloud computing systems) consistent with the disclosure. Alternatively, the software may be obtained and loaded (or, re-loaded/upgraded) from one or more servers and/or cloud computing systems, such as software stored on a server for distribution over the Internet, for example.

Method examples or other operations described herein can be machine or device (e.g., computer, robotic) implemented at least in part. The components of the illustrative devices, systems and methods employed in accordance with the illustrated embodiments may be implemented, at least in part, in digital electronic circuitry, analog electronic circuitry, or in computer hardware, firmware, software, or in combinations of them. These components may be implemented, for example, as a computing program product such as a computing program, program code or computer instructions tangibly embodied in an information carrier, or in a machine-readable storage device, for execution by, or to control the operation of, a data processing apparatus such as a programmable processor, a computer, or multiple computers. A computing program may be written in any form of programming language, including compiled or interpreted languages, and it may be deployed in any form, including as a stand-alone program or as a module, component, subroutine, or other unit suitable for use in a computing environment. Also, functional programs, codes, and code segments for accomplishing the techniques described herein may be easily construed as within the scope of the present disclosure by programmers skilled in the art. Method steps associated with the illustrative embodiments may be performed by one or more programmable processors executing a computing program, code or instructions to perform functions (e.g., by operating on input data and/or generating an output). Method steps may also be performed by, and apparatus may be implemented as, special purpose logic circuitry, e.g., an FPGA (field programmable gate array) or an ASIC (application-specific integrated circuit), for example.

Thus, in implementation in a controller or other machine for medical item processing, various logical blocks, modules, and circuits described in connection with the embodiments disclosed herein may be implemented or performed with a general-purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Processors suitable for the execution of a computing program include, by way of example, both general and special purpose microprocessors, and any one or more processors of any kind of digital computer. Information carriers suitable for embodying computing program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, etc.). The processor and the memory may be supplemented by or incorporated in special purpose logic circuitry.

As used herein, “machine-readable medium” or “machine-readable storage medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” or “machine-readable storage medium” should be taken to include a single medium or multiple media (e.g., a centralized or distributed database, or associated caches and servers) able to store processor instructions. The term “machine-readable medium” or “machine-readable storage medium” shall also be taken to include any medium, or combination of multiple media, which is capable of storing instructions for execution by one or more processors (or other processing circuitry), such that the instructions, when executed by one or more processors cause the one or more processors to perform any one or more of the methodologies described herein. Accordingly, a “machine-readable medium” or “machine-readable storage medium” refers to a single storage apparatus or device, as well as “cloud-based” storage systems or storage networks that include multiple storage apparatus or devices. A non-transitory “machine-readable medium” or “machine-readable storage medium” as used herein excludes signals per se.

Additional examples of the presently described embodiments include the following, non-limiting implementations. Each of the following non-limiting examples may stand on its own or may be combined in any permutation or combination with any one or more of the other examples provided below or throughout the present disclosure.

Example 1 is a method for normalizing spectrum to evaluate endoscope transmission, the method comprising: illuminating an entrance pupil of an endoscope with a first radiant flux, wherein the first radiant flux has a first spectrum; measuring a second radiant flux at an exit pupil of the endoscope, wherein the second radiant flux has a second spectrum; and determining a transmission measurement through the endoscope by comparing the first spectrum and the second spectrum.

In Example 2, the subject matter of Example 1 includes, wherein illuminating the entrance pupil comprises diffusing a light source through a first integrating sphere to cause the first radiant flux to overfill the entrance pupil of the endoscope.

In Example 3, the subject matter of Example 2 includes, wherein measuring the second radiant flux at the exit pupil comprises detecting light at an output of a second integrating sphere.

In Example 4, the subject matter of Example 3 includes, wherein detecting light at the output of the second integrating sphere comprises measuring the second spectrum at the output of the second integrating sphere.

In Example 5, the subject matter of Examples 1˜4 includes, wherein determining a transmission measure through the endoscope by comparing the first spectrum and the second spectrum comprises: determining a normalized first spectrum; determining a normalization factor; applying the normalization factor to the second spectrum to arrive at a normalized second spectrum; and determining the transmission measurement as a ratio of a first area in the normalized first spectrum to a second area in the normalized second spectrum, wherein the first area comprises a first sum of intensity values of the first spectrum in a first range of wavelengths, wherein the second area comprises a second sum of intensity values of the second spectrum in the first range of wavelengths, the first range of wavelengths occurring between and inclusive of a first wavelength and a second wavelength.

In Example 6, the subject matter of Example 5 includes, wherein the normalization factor for the second spectrum results in at least one intensity value at a given wavelength in the normalized second spectrum being approximately identical to at least one intensity value at the given wavelength in the first spectrum.

In Example 7, the subject matter of Examples 5-6 includes, wherein determining a normalization factor for the second spectrum comprises: determining a first maximum value of the first spectrum; normalizing the first spectrum by the first maximum value to arrive at the normalized first spectrum; determining a second maximum value of the second spectrum; normalizing the second spectrum by the second maximum value to create an intermediate second spectrum; determining the normalization factor as a third maximum value of a ratio of the intermediate second spectrum and the normalized first spectrum, wherein the third maximum value occurs at a third wavelength; and applying the normalization factor to the intermediate second spectrum to arrive at the normalized second spectrum, wherein a value of the normalized second spectrum at the third wavelength is approximately identical to a value of the first normalized spectrum at the third wavelength.

In Example 8, the subject matter of Examples 5-7 includes, wherein the first range of wavelengths is in a near-infrared portion of the electromagnetic spectrum.

In Example 9, the subject matter of Examples 1-8 includes, wherein the endoscope is a rigid endoscope.

In Example 10, the subject matter of Examples 1-9 includes, wherein the endoscope is a rigid near-infrared (NIR) endoscope.

In Example 11, the subject matter of Example 10 includes, wherein the rigid NIR endoscope is used for fluorescence endoscopy and wherein the transmission measurement is applied to a system for NIR imaging used in conjunction with the rigid NIR endoscope.

Example 12 is a non-transitory machine-readable storage medium comprising instructions, which when executed by circuitry of a machine, causes the circuitry to perform the operations of any one of Examples 1 to 11.

Example 13 is a system for normalizing spectrum to evaluate endoscope transmission, the system comprising: an endoscope having an entrance pupil and an exit pupil; a first light source diffused through a first integrating sphere, wherein a first integrating sphere is configured to illuminate the entrance pupil of the endoscope with a first spectrum of light; and a spectrometer configured to measure a second spectrum of light exiting a second integrating sphere, wherein the second integrating sphere is configured to collect light at the exit pupil of the endoscope; wherein the system is further configured to determine a transmission measurement through the endoscope by comparing the first spectrum and the second spectrum.

In Example 14, the subject matter of Example 13 includes, wherein diffusing the first light source through the first integrating sphere causes the first radiant flux to overfill the entrance pupil of the endoscope.

In Example 15, the subject matter of Examples 13-14 includes, wherein to determine the transmission measurement through the endoscope by comparing the first spectrum and the second spectrum, the system is further configured to: determine a normalized first spectrum; determine a normalization factor; apply the normalization factor to the second spectrum to arrive at a normalized second spectrum; and determine the transmission measurement as a ratio of a first area in the normalized first spectrum to a second area in the normalized second spectrum, wherein the first area comprises a first sum of intensity values of the first spectrum in a first range of wavelengths, wherein the second area comprises a second sum of intensity values of the second spectrum in the first range of wavelengths, the first range of wavelengths occurring between and inclusive of a first wavelength and a second wavelength.

In Example 16, the subject matter of Example 15 includes, wherein the normalization factor for the second spectrum results in at least one intensity value at a given wavelength in the normalized second spectrum being approximately identical to at least one intensity value at the given wavelength in the first spectrum.

In Example 17, the subject matter of Examples 15-16 includes, wherein to determine a normalization factor for the second spectrum, the system is further configured to: determine a first maximum value of the first spectrum; normalize the first spectrum by the first maximum value to arrive at the normalized first spectrum; determine a second maximum value of the second spectrum; normalize the second spectrum by the second maximum value to create an intermediate second spectrum; determine the normalization factor as a third maximum value of a ratio of the intermediate second spectrum and the normalized first spectrum, wherein the third maximum value occurs at a third wavelength; and apply the normalization factor to the intermediate second spectrum to arrive at the normalized second spectrum, wherein a value of the normalized second spectrum at the third wavelength is approximately identical to a value of the first normalized spectrum at the third wavelength.

In Example 18, the subject matter of Examples 15-17 includes, wherein the first range of wavelengths is in a near-infrared portion of the electromagnetic spectrum.

In Example 19, the subject matter of Examples 13-18 includes, wherein the endoscope is a rigid endoscope.

In Example 20, the subject matter of Examples 13-19 includes, wherein the endoscope is a rigid near-infrared (NIR) endoscope.

In Example 21, the subject matter of Example 20 includes, wherein the rigid NIR endoscope is used for fluorescence endoscopy and wherein the system is further configured to perform NIR imaging using the rigid NIR endoscope, and wherein the transmission measurement is applied to at least one NIR image.

Example 22 is at least one machine-readable medium including instructions that, when executed by processing circuitry, cause the processing circuitry to perform operations to implement of any of Examples 1-21.

Example 23 is an apparatus comprising means to implement of any of Examples 1-21.

Example 24 is a system to implement of any of Examples 1-21.

Example 25 is a method to implement of any of Examples 1-21.

What is claimed is:

1. A method for normalizing spectrum to evaluate endoscope transmission, the method comprising:
illuminating an entrance pupil of an endoscope with a first radiant flux, wherein the first radiant flux has a first spectrum;
measuring a second radiant flux at an exit pupil of the endoscope, wherein the second radiant flux has a second spectrum; and
determining a transmission measurement through the endoscope by comparing the first spectrum and the second spectrum.

2. The method of claim 1, wherein illuminating the entrance pupil comprises diffusing a light source through a first integrating sphere to cause the first radiant flux to overfill the entrance pupil of the endoscope.

3. The method of claim 2, wherein measuring the second radiant flux at the exit pupil comprises detecting light at an output of a second integrating sphere.

4. The method of claim 3, wherein detecting light at the output of the second integrating sphere comprises measuring the second spectrum at the output of the second integrating sphere.

5. The method of claim 1, wherein determining a transmission measure through the endoscope by comparing the first spectrum and the second spectrum comprises:
determining a normalized first spectrum;
determining a normalization factor;
applying the normalization factor to the second spectrum to arrive at a normalized second spectrum; and
determining the transmission measurement as a ratio of a first area in the normalized first spectrum to a second area in the normalized second spectrum, wherein the first area comprises a first sum of intensity values of the first spectrum in a first range of wavelengths, wherein the second area comprises a second sum of intensity values of the second spectrum in the first range of wavelengths, the first range of wavelengths occurring between and inclusive of a first wavelength and a second wavelength.

6. The method of claim 5, wherein the normalization factor for the second spectrum results in at least one intensity value at a given wavelength in the normalized second spectrum being approximately identical to at least one intensity value at the given wavelength in the first spectrum.

7. The method of claim 5, wherein determining a normalization factor for the second spectrum comprises:
determining a first maximum value of the first spectrum;
normalizing the first spectrum by the first maximum value to arrive at the normalized first spectrum;
determining a second maximum value of the second spectrum;
normalizing the second spectrum by the second maximum value to create an intermediate second spectrum;
determining the normalization factor as a third maximum value of a ratio of the intermediate second spectrum and the normalized first spectrum, wherein the third maximum value occurs at a third wavelength; and
applying the normalization factor to the intermediate second spectrum to arrive at the normalized second spectrum, wherein a value of the normalized second spectrum at the third wavelength is approximately identical to a value of the first normalized spectrum at the third wavelength.

8. The method of claim 5, wherein the first range of wavelengths is in a near-infrared portion of the electromagnetic spectrum.

9. The method of claim 1, wherein the endoscope is a rigid endoscope.

10. The method of claim 1, wherein the endoscope is a rigid near-infrared (NIR) endoscope.

11. The method of claim 10, wherein the rigid NIR endoscope is used for fluorescence endoscopy and wherein the transmission measurement is applied to a system for NIR imaging used in conjunction with the rigid NIR endoscope.

12. A non-transitory machine-readable storage medium comprising instructions for normalizing spectrum to evaluate endoscope transmission, which when executed by circuitry of a machine, cause the circuitry to:
illuminate an entrance pupil of an endoscope with a first radiant flux, wherein the first radiant flux has a first spectrum;
measure a second radiant flux at an exit pupil of the endoscope, wherein the second radiant flux has a second spectrum; and
determine a transmission measurement through the endoscope by comparing the first spectrum and the second spectrum.

13. A system for normalizing spectrum to evaluate endoscope transmission, the system comprising:
an endoscope having an entrance pupil and an exit pupil;
a first light source diffused through a first integrating sphere, wherein a first integrating sphere is configured to illuminate the entrance pupil of the endoscope with a first spectrum of light; and
a spectrometer configured to measure a second spectrum of light exiting a second integrating sphere, wherein the second integrating sphere is configured to collect light at the exit pupil of the endoscope;
wherein the system is further configured to determine a transmission measurement through the endoscope by comparing the first spectrum and the second spectrum.

14. The system of claim 13, wherein diffusing the first light source through the first integrating sphere causes a first radiant flux to overfill the entrance pupil of the endoscope.

15. The system of claim 13, wherein to determine the transmission measurement through the endoscope by comparing the first spectrum and the second spectrum, the system is further configured to:
determine a normalized first spectrum;
determine a normalization factor;
apply the normalization factor to the second spectrum to arrive at a normalized second spectrum; and
determine the transmission measurement as a ratio of a first area in the normalized first spectrum to a second area in the normalized second spectrum, wherein the first area comprises a first sum of intensity values of the first spectrum in a first range of wavelengths, wherein the second area comprises a second sum of intensity values of the second spectrum in the first range of wavelengths, the first range of wavelengths occurring between and inclusive of a first wavelength and a second wavelength.

16. The system of claim 15, wherein the normalization factor for the second spectrum results in at least one intensity value at a given wavelength in the normalized second spectrum being approximately identical to at least one intensity value at the given wavelength in the first spectrum.

17. The system of claim 15, wherein to determine a normalization factor for the second spectrum, the system is further configured to:
determine a first maximum value of the first spectrum;
normalize the first spectrum by the first maximum value to arrive at the normalized first spectrum;
determine a second maximum value of the second spectrum;
normalize the second spectrum by the second maximum value to create an intermediate second spectrum;
determine the normalization factor as a third maximum value of a ratio of the intermediate second spectrum and the normalized first spectrum, wherein the third maximum value occurs at a third wavelength; and
apply the normalization factor to the intermediate second spectrum to arrive at the normalized second spectrum, wherein a value of the normalized second spectrum at the third wavelength is approximately identical to a value of the first normalized spectrum at the third wavelength.

18. The system of claim 15, wherein the first range of wavelengths is in a near-infrared portion of the electromagnetic spectrum.

19. The system of claim 13, wherein the endoscope is a rigid endoscope.

20. The system of claim 13, wherein the endoscope is a rigid near-infrared (NIR) endoscope.

21. The system of claim 20, wherein the rigid NIR endoscope is used for fluorescence endoscopy and wherein the system is further configured to perform NIR imaging using the rigid NIR endoscope, and wherein the transmission measurement is applied to at least one NIR image.
